(19) United States
(12) Patent Application Publication
(10) Pub. No.: US 2016/0169996 A1
(43) Pub. Date: Jun. 16, 2016

(54) METHOD AND SYSTEM FOR CARDIAC SCAN PLANE PRESCRIPTION

(71) Applicant: General Electric Company, Schenectady, NY (US)

(72) Inventors: Hirohito Okuda, Yokohama (JP); Yoshihiro Oda, Hachioji (JP); Osamu Takayama, Hino (JP); Vivek Vaidya, Bangalore (IN)

(21) Appl. No.: 14/571,767

(22) Filed: Dec. 16, 2014

(51) Int. Cl. G01R 33/54; A61B 5/00; A61B 5/055

(52) U.S. Cl. CPC G01R 33/543; A61B 5/055; A61B 5/0044; A61B 5/7425; A61B 5/0042; A61B 5/4244; A61B 5/4528; A61B 2576/023; A61B 2560/0223; A61B 2576/026

(57) ABSTRACT

The present application discloses a method of prescribing a scan plane during Magnetic Resonance Imaging (MRI) of an anatomy. The method comprises acquiring a volume image of the anatomy at a first resolution and simultaneously displaying three planar images representing cross-sections through the anatomy. The three images are orthogonal to each other and intersecting at a common point in the volume. The method also comprises further displaying a pair of axes on each image showing a correspondence of the image with each of the other two images, and receiving a manipulation of one at least one of the axes on at least one of the images and automatically updating the other images to maintain the correspondences. The method also comprises acquiring a volume of the anatomy at a second resolution that is higher than the first resolution.

### BACKGROUND OF THE INVENTION

The subject matter disclosed herein relates to Magnetic Resonance Imaging (MRI), and more particularly, to a method of scan plane prescription for cardiac MRI.

For cardiac MRI scanning, a user typically acquires oblique plane images such as short-axis images, four-chamber images, and two-chamber images. Because the planes are oblique, neither normal nor parallel to any axis of mechanical coordinate system or patient coordinate system, a complex procedure is performed to determine each plane location and orientation. The procedure is time-consuming, typically taking more than several minutes. Additionally, the plane prescription procedure is difficult for a novice user to perform. It requires training and experience to do so competently and efficiently. Therefore, automation of scan plane orientation and location has been desired.

To address this problem, automatic scan plane computation techniques have been explored. These techniques typically start acquiring low resolution “localizer” images. In the case of cardiac imaging, the localizer image contains the whole heart volume in its field of view. The software then processes the input localizer image and computes scan plane location and orientation based on image processing techniques and machine learning.

However, there are known problems with this technology and method. For example, the success rate of automatically computing the correct plane prescription is not as high as desired. Achieving correct scan plane prescription at a success rate of 95% or higher is quite difficult. Additionally, the user is not aware of the failure of the scan plane prescription until after the higher resolution images are acquired, and manual manipulation of the higher resolution images is required. This can be very time consuming and difficult for novice users. Therefore, there is a need for a method and system to determine whether computed scan plane prescription is successful or not, and to correct plane prescription quickly and effectively when automatic scan plane prescription fails.

### BRIEF DESCRIPTION OF THE INVENTION

The above-mentioned shortcomings, disadvantages and problems are addressed herein which will be understood by reading and understanding the following specification.

In an embodiment a method of prescribing a scan plane during Magnetic Resonance Imaging (MRI) of an anatomy comprises acquiring a volume image of the anatomy at a first resolution and simultaneously displaying three planar images representing cross-sections through the anatomy. The three images are orthogonal to each other and intersecting at a common point in the volume. The method also comprises further displaying a pair of axes on each image showing a correspondence of the image with each of the other two images, and receiving a manipulation of one at least one of the axes on at least one of the images and automatically updating the other images to maintain the correspondences. The method also comprises acquiring a volume of the anatomy at a second resolution that is higher than the first resolution.

In another embodiment, a MRI system comprises an apparatus configured to acquire an MRI volume image of an anatomy and a processing unit configured to prescribe a scan plane. The system also comprises an operator console comprising a display and an input device. The display is configured to display the prescribed scan plane comprising three planar images representing cross-sections through the anatomy, the three images being orthogonal to each other and intersecting at a common point in the volume, and a pair of axes on each image showing a correspondence of the image with each of the other two images. The processing unit is configured to receive manipulation of at least one of the axes on at least one of the images by the input device in order to modify the prescribed scan plane.

Various other features, objects, and advantages of the invention will be made apparent to those skilled in the art from the accompanying drawings and detailed description thereof.

### BRIEF DESCRIPTION OF THE DRAWINGS

FIG. 1 is a schematic block diagram of an exemplary magnetic resonance imaging (MRI) system in accordance with an embodiment of the disclosure;

FIG. 2 is a schematic representation of a screen shot in accordance with an embodiment of the disclosure; and

FIG. 3 is a flowchart of a method of prescribing a scan plane during MRI of an anatomy in accordance with an embodiment of the disclosure.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the invention.

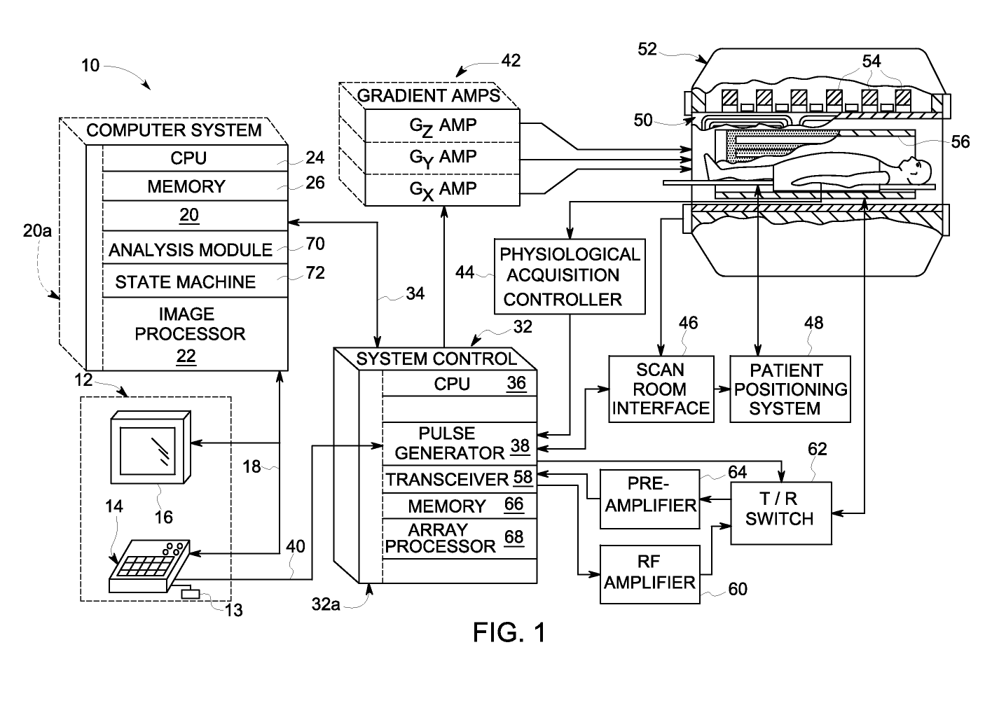

FIG. 1 is a schematic block diagram of an exemplary magnetic resonance imaging (MRI) system in accordance with an embodiment. The operation of MRI system 10 is controlled from an operator console 12 that includes a keyboard or other input device 13, a control panel 14, and a display 16. The console 12 communicates through a link 18 with a computer system 20 and provides an interface for an operator to prescribe MRI scans, display resultant images, perform image processing on the images, and archive data and images. The computer system 20 includes a number of modules that communicate with each other through electrical and/or data connections, for example, such as are provided by using a backplane 20a. Data connections may be direct wired links or may be fiber optic connections or wireless communication links or the like. The modules of the computer system 20 include an image processor module 22, a CPU module 24 and a memory module 26 which may include a frame buffer for storing image data arrays. In an alternative embodiment, the image processor module 22 may be replaced by image processing functionality on the CPU module 24. The computer system 20 is linked to archival media devices, permanent or back-up memory storage or a network. Computer system 20 may also communicate with a separate system control computer 32 through a link 34. The input device 13 can include a mouse, joystick, keyboard, track ball, touch activated screen, light wand, voice control, or any similar or equivalent input device, and may be used for interactive geometry prescription.

The system control computer 32 includes a set of modules in communication with each other via electrical and/or data connections 32a. Data connections 32a may be direct wired links, or may be fiber optic connections or wireless communication links or the like. In alternative embodiments, the modules of computer system 20 and system control computer 32 may be implemented on the same computer system or a plurality of computer systems. The modules of system control computer 32 include a CPU module 36 and a pulse generator module 38 that connects to the operator console 12 through a communications link 40. The pulse generator module 38 may alternatively be integrated into the scanner equipment (e.g., resonance assembly 52). It is through link 40 that the system control computer 32 receives commands from the operator to indicate the scan sequence that is to be performed. The pulse generator module 38 operates the system components that play out (i.e., perform) the desired pulse sequence by sending instructions, commands and/or requests describing the timing, strength and shape of the RF pulses and pulse sequences to be produced and the timing and length of the data acquisition window. The pulse generator module 38 connects to a gradient amplifier system 42 and produces data called gradient waveforms that control the timing and shape of the gradient pulses that are to be used during the scan. The pulse generator module 38 may also receive patient data from a physiological acquisition controller 44 that receives signals from a number of different sensors connected to the patient, such as ECG signals from electrodes attached to the patient. The pulse generator module 38 connects to a scan room interface circuit 46 that receives signals from various sensors associated with the condition of the patient and the magnet system. It is also through the scan room interface circuit 46 that a patient positioning system 48 receives commands to move the patient table to the desired position for the scan.

The gradient waveforms produced by the pulse generator module 38 are applied to gradient amplifier system 42 which is comprised of Gₓ, G_{y} and G_{z} amplifiers. Each gradient amplifier excites a corresponding physical gradient coil in a gradient coil assembly generally designated 50 to produce the magnetic field gradient pulses used for spatially encoding acquired signals. The gradient coil assembly 50 forms part of a resonance assembly 52 that includes a polarizing superconducting magnet with superconducting main coils 54. Resonance assembly 52 may include a whole-body RF coil 56, surface or parallel imaging coils 76 or both. The coils 56, 76 of the RF coil assembly may be configured for both transmitting and receiving or for transmit-only or receive-only. A patient or imaging subject 70 may be positioned within a cylindrical patient imaging volume 72 of the resonance assembly 52. A transceiver module 58 in the system control computer 32 produces pulses that are amplified by an RF amplifier 60 and coupled to the RF coils 56, 76 by a transmit/receive switch 62. The resulting signals emitted by the excited nuclei in the patient may be sensed by the same RF coil 56 and coupled through the transmit/receive switch 62 to a preamplifier 64. Alternatively, the signals emitted by the excited nuclei may be sensed by separate receive coils such as parallel coils or surface coils 76. The amplified MR signals are demodulated, filtered and digitized in the receiver section of the transceiver 58. The transmit/receive switch 62 is controlled by a signal from the pulse generator module 38 to electrically connect the RF amplifier 60 to the RF coil 56 during the transmit mode and to connect the preamplifier 64 to the RF coil 56 during the receive mode. The transmit/receive switch 62 can also enable a separate RF coil (for example, a parallel or surface coil 76) to be used in either the transmit or receive mode.

The MR signals sensed by the RF coil 56 or parallel or surface coil 76 are digitized by the transceiver module 58 and transferred to a memory module 66 in the system control computer 32. Typically, frames of data corresponding to MR signals are stored temporarily in the memory module 66 until they are subsequently transformed to create images. An array processor 68 uses a known transformation method, most commonly a Fourier transform, to create images from the MR signals. These images are communicated through the link 34 to the computer system 20 where it is stored in memory. In response to commands received from the operator console 12, this image data may be archived in long-term storage or it may be further processed by the image processor 22 and conveyed to the operator console 12 and presented on display 16.

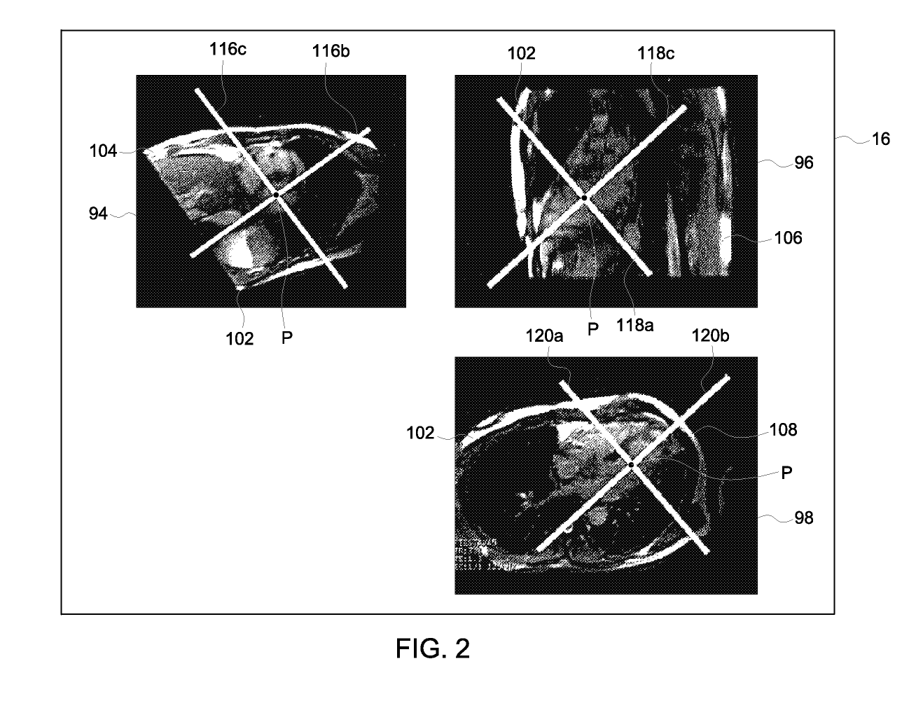

Referring to FIG. 2, the display 16 is depicted in accordance with embodiment. The display 16 comprises three planar images 104, 106, 108 of an anatomy 102. The anatomy 102 will hereinafter be described as a heart. It should be appreciated, however, that other anatomies may be envisioned. For example, the anatomy 102 may be a liver, a brain or a joint.

In the depicted embodiment, each image 104, 106, 108 is comprised in a viewport 94, 96, 98, respectively. It should be appreciated that the distinct viewports 94, 96, 98 for each image 104, 106, 108 could be eliminated while also operating within the scope of the present disclosure. For example, all three images 104, 106, 108 may be comprised in a single viewport.

Each planar image 104, 106, 108 represents a cross-section through the heart 102. For example, image 104 represents a short-axis view of the heart 102. The short-axis view depicts the left ventricle and the right ventricle. In another example, image 106 represents a 2-chamber view of the heart 102. The 2-chamber view depicts the left ventricle and the left atrium. In yet another example, image 108 represents the 4-chamber view of the heart 102 and it depicts the left ventricle, left atrium, right ventricle and the right atrium.

Each of the three images 104, 106, 108, are orthogonal to each other and intersect at a common point P in the volume of the anatomy 102. For the purpose herein, the term orthogonal may be defined as perpendicular. For example, three planes that are orthogonal to one another may be the axial, sagittal and coronal planes, wherein the axial plane divides the superior and inferior parts of the body, the sagittal plane divides the right side of the body from the left, and the coronal plane divides the anterior (front) of the body from the posterior (back) of the body.

A pair of axes 116, 118, 120, may be superimposed on each image 104, 106, 108, respectively. The pair of axes 116 comprises axis 116b and axis 116c. The pair of axes 118 comprises axis 118a and axis 118c. The pair of axes 120 comprises axis 120a and axis 120b. The pairs of axes 116, 118, 120 show a correspondence of the image with each of the other two images. For example, image 104 has axis 116b which represents the intersection of the plane of image 104 with the plane of image 106. Axis 116c represents the intersection of the plane of image 104 with the plane of image 108. In another example, image 106 has axis 118a which represents the intersection of the plane of image 106 with the plane of image 104. Axis 118c represents the intersection of the plane of image 106 with the plane of image 108. In yet another example, image 108 has axis 120a which represents the intersection of the plane of image 108 with the plane of image 104. Axis 120b represents the intersection of the plane of image 108 with the plane of image 106.

It may be desirable to use a color-coding scheme to quickly and efficiently aid the user to identify the corresponding axes with image or viewport. In one embodiment, the viewports 94, 96, 98 and/or images 104, 106, 108 are color coded and the axes 116b, 116c, 118a, 118c, 120a, 120b are colored to correspond to the intersecting image. The viewports 94, 96, 98 and/or images 104, 106, 108 may be color coded in a variety of ways. For example, in one embodiment, the viewport 94 may be outlined in a color such as red, for example, and axes 118a and 120a would also be colored red to show a correspondence to viewport 94. In another embodiment, there is a blue-colored symbol, for example, in viewport 96 and axes 116b and 120b would also be colored blue. In yet another embodiment, text appearing in viewport 98 may be in a green-colored font and axes 116c and 118c would be in green. It should be appreciated that other embodiments of the color-coding of viewports 94, 96, 98 and/or images 104, 106, 108 with axes 116b, 116c, 118a, 118c, 120a, 120b may be envisioned.

Each axis 116b, 116c, 118a, 118c, 120a, 120b may be manipulated via the input device 13. The manipulation may include rotating, shifting or a combination thereof. Rotating may comprise the movement of one axis about the intersection point P. Shifting may comprise movement of one axis such that the intersection point P is modified.

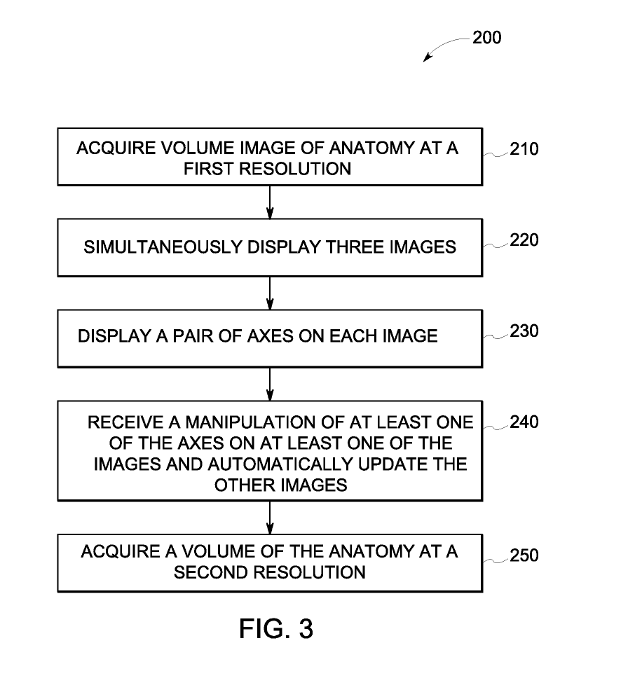

Having described exemplary components of the MRI system 10, a method 200 of prescribing a scan plane will now be described in accordance with an embodiment. Referring to FIG. 3, the method 200 may include a step 210 comprising acquiring a volume image of the anatomy 102 at a first resolution. As stated above with respect to FIG. 2, the anatomy 102 may be a heart. It should be appreciated, however, that other anatomies may be envisioned. For example, the anatomy 102 may be a liver, a brain or a joint. The volume image may be a “localizer” image as is commonly known in the art. At step 210, the first resolution may be a low resolution. For example, in one embodiment, the first resolution may be between 1.5 mm and 8 mm.

The method 200 may include a step 220 comprising simultaneously displaying the three planar images 104, 106, 108 on the display 16. For purposes herein, simultaneously may be defined as concurrent. As described above with respect to FIG. 2, the three images 104, 106, 108 are orthogonal to each other and intersect at an intersection point P in the volume of the anatomy 102. In one embodiment, the three planar images 104, 106, 108 may be a short-axis image, a 2-chamber image and a 4-chamber image, respectively. The three images 104, 106, 108 may be displayed in viewports 94, 96, 98, respectively, or, alternatively, the images 104, 106, 108 may be displayed in another configuration or manner.

The method 200 may include a step 230 comprising displaying the pair of axes 116, 118, 120 on each image 104, 106, 108. The pairs of axes 116, 118, 120 may be superimposed on each image 104, 106, 108. The pair of axes 116 comprises axis 116b and axis 116c. The pair of axes 118 comprises axis 118a and axis 118c. The pair of axes 120 comprises axis 120a and axis 120b.

Each pair of axes 116, 118, 120 represents a correspondence of the image with the other two images. For example, image 104 has axis 116b which represents the intersection of the plane of image 104 with the plane of image 106. Axis 116c represents the intersection of the plane of image 104 with the plane of image 108. In another example, image 106 has axis 118a which represents the intersection of the plane of image 106 with the plane of image 104. Axis 118c represents the intersection of the plane of image 106 with the plane of image 108. In yet another example, image 108 has axis 120a which represents the intersection of the plane of image 108 with the plane of image 104. Axis 120b represents the intersection of the plane of image 108 with the plane of image 106.

In one embodiment, the images and/or viewports may be color coded with the axes 116b, 116c, 118a, 118c, 120a, 120b may be color-coded in order to show the correspondences between axes and images.

The method 200 may include a step 240 comprising receiving manipulation of at least one of the axes 116b, 116c, 118a, 118c, 120a, 120b on at least one of the images 104, 106, 108, and automatically updating the other images to maintain correspondences. Each axis 116b, 116c, 118a, 118c, 120a, 120b may be manipulated via the input device 13. The manipulation may include rotating, shifting or a combination thereof. Rotating may comprise the movement of one axis about the intersection point P. Shifting may comprise the movement of one axis such that the intersection point P is altered.

In one embodiment, the step 240 may be performed substantially in real time. For the purpose herein, substantially in real time may be defined as without intentional delay. For example, the images may be updated within one to two seconds of receipt of the manipulation.

The method 200 may include a step 250 comprising acquiring a volume of the anatomy 102 at a second resolution that is higher than the first resolution. In one embodiment, the second resolution may be between 1 mm and 2 mm.

The method 200 and apparatus 10 provide numerous benefits. For example, the refinement and adjustment of an automatically calculated scan plane allows for more efficient, faster, scan plane prescription, especially where the automatically calculated prescription fails. It also makes scan plane prescription more accessible for novice users as it eliminates the need for entirely manual prescription.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

We claim:

1. A method of prescribing a scan plane during Magnetic Resonance Imaging (MRI) of an anatomy comprising:
acquiring a volume image of the anatomy at a first resolution;
simultaneously displaying three planar images representing cross-sections through the anatomy, the three images being orthogonal to each other and intersecting at a common point in the volume,
further displaying a pair of axes on each image showing a correspondence of the image with each of the other two images,
receiving a manipulation of one at least one of the axes on at least one of the images, and automatically updating the other images to maintain the correspondences, and
acquiring a volume of the anatomy at a second resolution that is higher than the first resolution.

2. The method of claim 1, wherein the anatomy is a heart.

3. The method of claim 2, wherein the three planar images comprise a 4-chamber image, a 2-chamber image and a short-axis image.

4. The method of claim 1, wherein each planar image is displayed in a viewport.

5. The method of claim 1, wherein the manipulation is rotating.

6. The method of claim 1, wherein the manipulation is shifting.

7. The method of claim 1, wherein the receiving a manipulation and updating the other images step occurs substantially in real time.

8. The method of claim 1, further comprising acquiring a calibration scan at a scan location in order to align the scan location with the anatomy.

9. The method of claim 1, wherein each image is color coded and the axis corresponding to that image is similarly colored.

10. The method of claim 1, wherein the anatomy is a liver.

11. The method of claim 1, wherein the anatomy is a brain.

12. The method of claim 1, wherein the anatomy is a joint.

13. A Magnetic Resonance Imaging (MRI) system comprising:
an apparatus configured to acquire an MRI volume image of an anatomy;
a processing unit configured to prescribe a scan plane; and
an operator console comprising a display and an input device,
wherein the display is configured to display the prescribed scan plane comprising three planar images representing cross-sections through the anatomy, the three images being orthogonal to each other and intersecting at a common point in the volume, and a pair of axes on each image showing a correspondence of the image with each of the other two images; and
wherein the processing unit is configured to receive manipulation of at least one of the axes on at least one of the images by the input device in order to modify the prescribed scan plane.

14. The MRI system of claim 13, wherein the processing unit is further configured to update all three planar images substantially in real time based on the manipulation in order to maintain correspondences.

15. The MRI system of claim 13, wherein the anatomy is a heart.

16. The MRI system of claim 15, wherein the three planar images comprises a 4-chamber image, a 2-chamber image and a short-axis image.

17. The MRI system of claim 13, wherein the manipulation is rotating.

18. The MRI system of claim 13, wherein the manipulation is shifting.

19. The MRI system of claim 13, wherein each image is color coded and the axis corresponding to that image is similarly colored.

20. The MRI system of claim 13, wherein the anatomy is at least one of a liver, brain, and joint.
